(19) United States
(12) Patent Application Publication
(10) Pub. No.: US 2018/0353380 A1
(43) Pub. Date: Dec. 13, 2018

(54) ASSEMBLY AND ADAPTER FOR REGULATING PRESSURE DURING FLUID TRANSER

(71) Applicant: Critical Care Excellence, LLC, Goldsboro, NC (US)

(72) Inventors: Michael Carr Berrier, Goldsboro, NC (US); Jared Little, Durham, NC (US)

(21) Appl. No.: 15/619,639

(22) Filed: Jun. 12, 2017

(51) Int. Cl. A61J 1/20; A61M 5/31

(52) U.S. Cl. CPC A61J 1/2072; A61M 5/162; A61M 5/3134; A61J 1/20

(57) ABSTRACT

The subject matter described herein includes an assembly having a syringe, a pressure gauge, and an adapter. The adapter defines a longitudinal pathway that allows for flow through of fluids from a first terminal inlet to a second terminal outlet. The adapter also defines a second pathway that allows for flow through of fluids from a third terminal inlet into the longitudinal pathway. The first terminal inlet defines a threaded portion that selectively engages the syringe. The adapter defines a knurled grip that facilitates stabilization of the adapter when engaged with the syringe. The second terminal outlet selectively engages a tube that carries an inflatable balloon on an end thereof. The balloon is configured for sealing a patient's trachea when inflated. The third terminal inlet selectively engages the pressure gauge.

### TECHNICAL FIELD

The presently disclosed subject matter relates to methods and devices related to improvements in the use of a medical breathing tube. In some embodiments, the invention reduces injuries and diseases associated with the use of the breathing tube. In one embodiment, the present invention provides methods and devices to ensure proper pressure of an inflatable balloon inserted in a patient to seal their trachea or esophagus. The presently disclosed subject matter is directed to an assembly and adapter for regulation pressure during fluid transfer.

### BACKGROUND

Tracheal intubation is one of the most common procedures performed in an operating room and during transit in an ambulance. The procedure involves the introduction of a plastic endotracheal tube into the patient's trachea to create an open channel for air and gas to flow. An inflatable balloon sits at the distal end of the tube. The balloon inflates when pumped with air, ideally creating an airtight seal between the tube and trachea.

A common process involving a medical tube is endotracheal intubation whereby a length of medical tubing is inserted into a human patient's trachea to provide access to the lungs. The tubing may be inserted orally, nasally or through another opening, such as a tracheostomy. A gas is passed through the tubing to the lungs. An inflatable balloon located circumferentially around the tubing exerts pressure on an interior wall of a body passage. The presence of the balloon segregates the upper airway structures, reducing fluid leaking from or to the lungs, thus improving the effectiveness of the gas exchange.

The pressure created by air pumped into the balloon is known as the “cuff pressure”. Maintaining adequate cuff pressure is critical for several reasons. It ensures that an airtight seal is created that allow for effective oxygen and gas transfer. Also, the physical cuff prevents patient aspiration by restricting the buildup of mucous or vomit from accessing the lungs.

While it is critical that balloons are inflated sufficiently, only a narrow range of pressure values (15-35 cm H₂O) is considered safe and appropriate. Pressures slightly above this range result in numerous physiological injuries such as the impedance of tracheal blood flow, tracheal damage, tissue damage, tracheomalacia, and floppy trachea. For example, at high pressures, blood flow at the cuff site can be reduced significantly allowing for pathologic changes including ischemia, inflammation, ulceration, granulation, and stenosis.

The current paradigm toward maintaining adequate cuff pressure relies on a subjective finger palpation method, in which a pilot balloon attached to the distal end of the ET tube is “pinched” by hand to assess the adequacy of pressure. This “pinch” method is known to be highly imprecise in clinical practice.

Maintaining cuff pressures is important. Excessively high pressure in the cuff causes tracheal wall injury, while low pressure allows fluids to flow down the trachea into the lung and may result in diseases and disorders including but not limited to ventilator-associated pneumonia. The aforementioned disorders are difficult to treat and may result in death. Thus, there is a need to measure and correctively regulate tracheal tube cuff pressure.

A device called a supraglottic, extraglottic, or blind insertion airway device is often used to seal the esophagus of a patient. In these devices the distal balloon rests in the esophagus and the proximal balloon seals the upper throat or oropharnyx. The device is labeled with an inflation amount, which varies by size, but not a safe inflation pressure. Overfilling this devices has similar or worse deleterious effects as the ET tube.

While the technology of the prior art disclose various methods of obtaining and maintaining therapeutic pressures, they fail to teach an adapter that is capable of retrofitting a traditional breathing assistance assembly with a manometer. There is a need for a re-usable, transferable adapter that can be used with traditional breathing assistance assemblies carried by most first responders.

### SUMMARY

This summary is provided to introduce in a simplified form concepts that are further described in the following detailed descriptions. This summary is not intended to identify key features or essential features of the claimed subject matter, nor is it to be construed as limiting the scope of the claimed subject matter.

At least one embodiment of the presently disclosed subject matter includes an assembly having a syringe, a pressure gauge, and an adapter. The adapter defines a longitudinal pathway that allows for flow through of fluids from a first terminal inlet to a second terminal outlet and a second pathway that allows for flow through of fluids from a third terminal inlet into the longitudinal pathway.

The first terminal inlet defines a threaded portion that selectively engages the syringe when in an operational mode and disengages when in a storage mode. The adapter defines a knurled grip that facilitates stabilization of the adapter when engaged with the syringe.

The second terminal outlet selectively engages a tube, when in the operational mode, that carries an inflatable balloon on an end thereof. The balloon is configured for sealing a patient's trachea when inflated.

The tube is disengaged from the second terminal outlet when in the storage mode. The third terminal inlet selectively engages the pressure gauge when in the operational mode and disengages the pressure gauge when in the storage mode.

In the operational mode a user engages the first terminal inlet to the syringe, the second terminal outlet to the tube, the third terminal inlet to the pressure gauge, and the balloon is placed in the patient's trachea and inflated by an original actuation of the syringe.

Some embodiments of the presently disclosed subject matter may include the assembly where the second pathway is positioned relative to the longitudinal pathway at an angle which is greater than zero degrees and less than 180 degrees.

The presently disclosed subject matter may include the assembly where pressure is released from the balloon by actuation of the syringe in a direction opposite from a direction of the original actuation of the syringe.

The assembly may also include a suction port carried by and extending from the tube. The pressure gauge may include a display for displaying a pressure to the user. The presently disclosed subject matter may include the assembly where the longitudinal pathway is rigid such that movement of the syringe imparts corresponding movement of the adapter.

One embodiment of the presently disclosed subject matter may include the assembly where the tube carries a second inflatable balloon that is configured for sealing a patient's esophagus when inflated.

At least one embodiment of the presently disclosed subject matter includes an adapter having a longitudinal pathway that allows for flow through of fluids from a first terminal inlet to a second terminal outlet. The adapter includes a second pathway that allows for flow through of fluids from a third terminal inlet into the longitudinal pathway.

The first terminal inlet defines a threaded portion that selectively engages a syringe when in an operational mode and disengages when in a storage mode. The adapter defines a knurled grip that facilitates stabilization of the adapter when engaged with the syringe.

The second terminal outlet selectively engages a tube, when in the operational mode, that carries an inflatable balloon on an end thereof. The balloon is configured for sealing a patient's trachea when inflated.

The tube is disengaged from the second terminal outlet when in the storage mode. The third terminal inlet selectively engages a pressure gauge when in the operational mode and disengages the pressure gauge when in the storage mode.

In the operational mode a user engages the first terminal inlet to the syringe, the second terminal outlet to the tube, the third terminal inlet to the pressure gauge, and the balloon is placed in the patient's trachea and inflated by an original actuation of the syringe.

One embodiment of the presently disclosed subject matter includes a method of inflating a balloon. The method includes using an adapter that defines a longitudinal pathway which allows for flow through of fluids from a first terminal inlet to a second terminal outlet. The adapter also defines a second pathway that allows for flow through of fluids from a third terminal inlet into the longitudinal pathway.

The method also includes engaging a syringe with a threaded portion at the first terminal inlet. The adapter defines a knurled grip that facilitates stabilization of the adapter when engaged with the syringe.

The method includes engaging a tube with the second terminal outlet. The tube carries an inflatable balloon on an end thereof. The balloon is configured for sealing a patient's trachea when inflated.

The method also includes engaging a pressure gauge with the third terminal inlet, placing the balloon in the patient's trachea, actuating the syringe to inflate the balloon, and checking the pressure of the inflated balloon using the pressure gauge.

In some embodiments of the presently disclosed subject matter the method may also include the second pathway positioned relative to the longitudinal pathway at an angle which is greater than zero degrees and less than 180 degrees.

The method may further include determining if pressure needs to be released from the balloon, and actuating the syringe in a direction opposite from a direction of the original actuation of the syringe to release pressure from the balloon.

The method may also include a suction port carried by and extending from the tube. The method may include a pressure gauge having a display for displaying a pressure to the user. The method may also include a rigid longitudinal pathway such that movement of the syringe imparts corresponding movement of the adapter.

The method may include a tube carrying a second inflatable balloon, wherein the second balloon is configured for sealing a patient's esophagus when inflated.

The method may further include disengaging the syringe from the first terminal inlet, disengaging the tube from the second terminal outlet, and disengaging the pressure gauge from the third terminal inlet when in the storage mode.

### BRIEF DESCRIPTION OF THE DRAWINGS

The foregoing, as well as the following Detailed Description of preferred embodiments, is better understood when read in conjunction with the appended drawings. For the purposes of illustration, there is shown in the drawings exemplary embodiments; however, the presently disclosed subject matter is not limited to the specific methods and instrumentalities disclosed.

FIG. 1 is a top view of an adapter according to one or more embodiments of the presently disclosed subject matter.

FIG. 2 is a perspective view of the adapter according to one or more embodiments of the presently disclosed subject matter.

FIG. 3 is a front facing view of an assembly in the operational mode according to one or more embodiments of the presently disclosed subject matter.

FIG. 4 is a front facing view of the assembly in the storage mode according to one or more embodiments of the presently disclosed subject matter.

FIG. 5 is a perspective view of the assembly where a tube is carrying a second inflatable balloon and a suction port according to one or more embodiments of the presently disclosed subject matter.

FIG. 6 is a view of the assembly showing a balloon placed in a patient's trachea according to one or more embodiments of the presently disclosed subject matter.

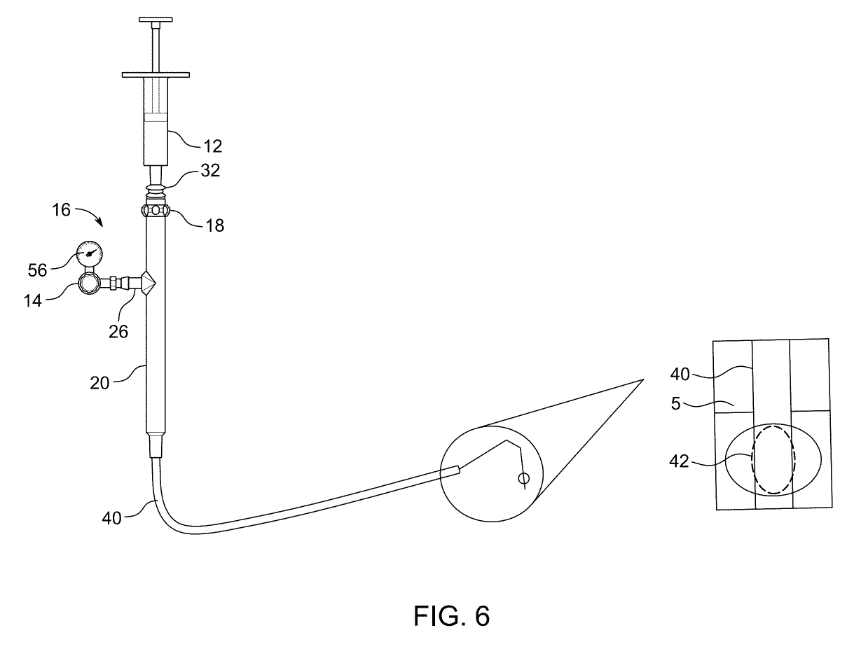

### DETAILED DESCRIPTION

These descriptions are presented with sufficient details to provide an understanding of one or more particular embodiments of broader inventive subject matters. These descriptions expound upon and exemplify particular features of those particular embodiments without limiting the inventive subject matters to the explicitly described embodiments and features. Considerations in view of these descriptions will likely give rise to additional and similar embodiments and features without departing from the scope of the inventive subject matters. Although the term “step” may be expressly used or implied relating to features of processes or methods, no implication is made of any particular order or sequence among such expressed or implied steps unless an order or sequence is explicitly stated.

Any dimensions expressed or implied in the drawings and these descriptions are provided for exemplary purposes. Thus, not all embodiments within the scope of the drawings and these descriptions are made according to such exemplary dimensions. The drawings are not made necessarily to scale. Thus, not all embodiments within the scope of the drawings and these descriptions are made according to the apparent scale of the drawings with regard to relative dimensions in the drawings. However, for each drawing, at least one embodiment is made according to the apparent relative scale of the drawing.

Unless defined otherwise, all technical and scientific terms used herein have the same meaning as commonly understood to one of ordinary skill in the art to which the presently disclosed subject matter pertains. Although any methods, devices, and materials similar or equivalent to those described herein can be used in the practice or testing of the presently disclosed subject matter, representative methods, devices, and materials are now described.

Following long-standing patent law convention, the terms “a”, “an”, and “the” refer to “one or more” when used in the subject specification, including the claims. Thus, for example, reference to “a device” can include a plurality of such devices, and so forth.

Unless otherwise indicated, all numbers expressing quantities of components, conditions, and so forth used in the specification and claims are to be understood as being modified in all instances by the term “about”. Accordingly, unless indicated to the contrary, the numerical parameters set forth in the instant specification and attached claims are approximations that can vary depending upon the desired properties sought to be obtained by the presently disclosed subject matter.

As used herein, the term “about”, when referring to a value or to an amount of mass, weight, time, volume, concentration, and/or percentage can encompass variations of, in some embodiments +/−20%, in some embodiments +/−10%, in some embodiments +/−5%, in some embodiments +/−1%, in some embodiments +/−0.5%, and in some embodiments +/−0.1%, from the specified amount, as such variations are appropriate in the presently disclosed subject matter.

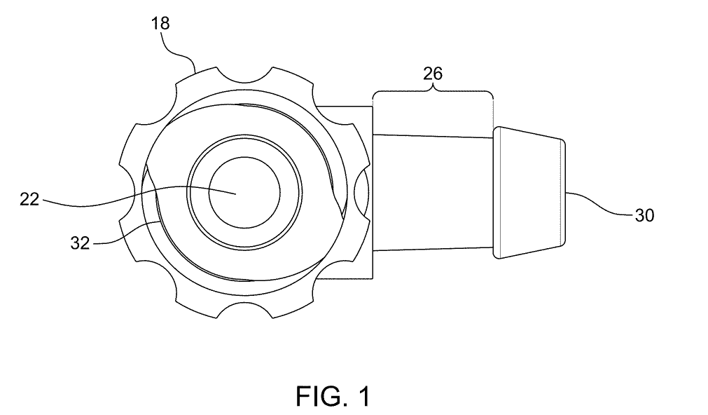

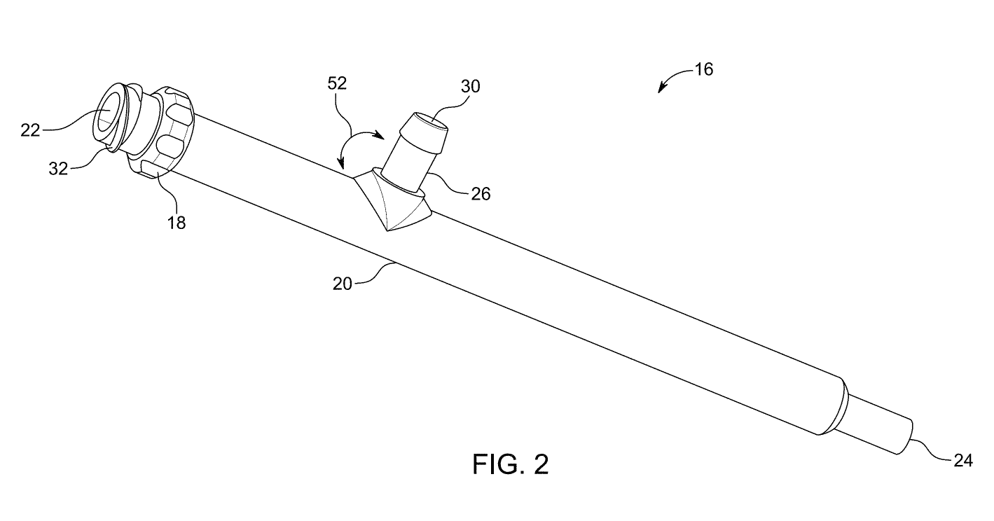

At least one embodiment of the presently disclosed subject matter is illustrated throughout the drawings and in particular reference to FIGS. 1 and 2. Disclosed herein is an assembly 10 having a syringe 12, a pressure gauge 14, and an adapter 16. The adapter 16 defines a longitudinal pathway 20 that allows for flow through of fluids from a first terminal inlet 22 to a second terminal outlet 24. The adapter 16 also defines a second pathway 26 that allows for flow through of fluids from a third terminal inlet 30 into the longitudinal pathway 20.

The first terminal inlet 22 defines a threaded portion 32 that selectively engages the syringe 12 when in an operational mode and disengages when in a storage mode. A bulb inflator may be used as a substitution for the syringe 12. The first terminal inlet 22 may define a threaded portion 32 or any connecting portion. The connecting portion can include threads, snap fit closures or any mechanism to create a tight seal with an end of the syringe 12. The syringe 12 may engage with the threaded portion 32 through the use of a luer lock fitting. In some embodiments, the adapter 16 defines a knurled grip 18 that facilitates stabilization of the adapter 16 when engaged with the syringe 12.

The second terminal outlet 24 selectively engages a tube 40 when in the operational mode. The tube 40 can be made of any material known or used in the art including, but not limited to, polymeric material, rubber, or combination thereof. The end of the tube 40 carries an inflatable balloon 42. The balloon 42 is configured for sealing a patient's trachea 5 when inflated. Thus, the diameter of the balloon 42 can be sized to be the same or about the same diameter as the patient's trachea 5. The balloon 42 can include any suitable inflatable device known or used in the medical field and can be constructed from any material known or used in the art including, but not limited to, polymeric material, rubber, or combinations thereof. The tube 40 is disengaged from the second terminal outlet 24 when in the storage mode. The third terminal inlet 30 selectively engages the pressure gauge 14 when in the operational mode and disengages the pressure gauge 14 when in the storage mode. The pressure gauge 14 can be any device capable of measuring and displaying the pressure within pathway 20, including, but not limited to, mechanical pressure gauges, pressure sensors, and the like.

In the operational mode a user engages the first terminal inlet 22 to the syringe 12 using any method known in the art. For example, in some embodiments, the syringe can be releasably attached to the first terminal inlet using a snap fit closure, fasteners, mechanical closures, threaded fit closure, and the like. A luer lock fitting may be used to attach the syringe 12 to the first terminal inlet 22. In the operational mode, the second terminal outlet 24 is connected to the tube 40 using any method known in the art (e.g. sliding the terminal outlet 24 into the tube 40). Further, in the operational mode, the third terminal inlet 30 is connected to the pressure gauge 14 using any method known in the art, such as, but not limited to, using a snap fit closure, fasteners, mechanical closures, threaded fit closure, and the like. Balloon 42 is then placed in the patient's trachea 5 by a user and inflated by an original actuation of the syringe 12. Particularly, the balloon is inflated by repeatedly depressing the plunger of the syringe until the balloon is inflated to a desired level.

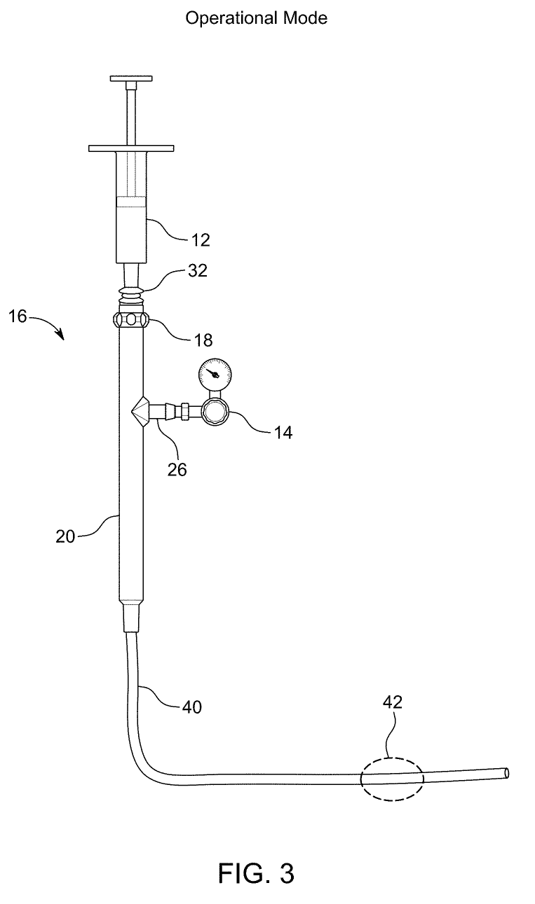

One embodiment of the operational mode is shown in FIG. 3. In this mode, the first terminal inlet 22 defines a threaded portion 32 that selectively engages a syringe 12. The adapter 16 has a knurled grip 18 that facilitates stabilization of the adapter 16 when engaged with the syringe 12. In this mode, the second terminal outlet 24 selectively engages a tube 40 that carries an inflatable balloon 42 on an end thereof. The balloon 42 is configured for sealing a patient's trachea 5 when inflated. The third terminal inlet 30 selectively engages a pressure gauge 14 when in the operational mode. In this mode, a user engages the first terminal inlet 22 to the syringe 12, the second terminal outlet 24 to the tube 40, the third terminal inlet 30 to the pressure gauge 14, and the balloon 42 is placed in the patient's trachea 5 and inflated by an original actuation of the syringe 12.

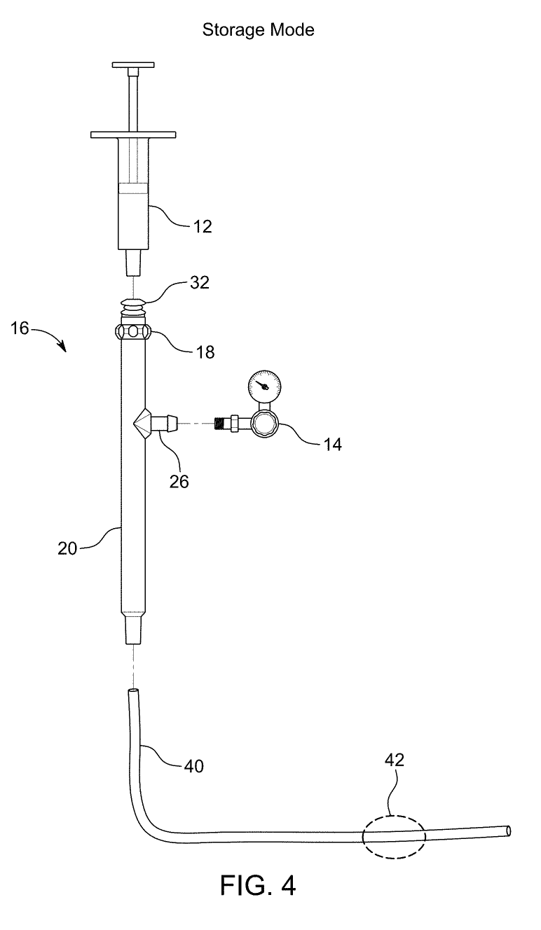

One embodiment of the storage mode is shown in FIG. 4. In this mode, the first terminal inlet 22 defines a threaded portion 32 that disengages from the syringe 12 when in the storage mode. The tube 40 is disengaged from the second terminal outlet 24 when in the storage mode. The third terminal inlet 30 disengages the pressure gauge 14 when in the storage mode. In the storage mode a user disengages the first terminal inlet 22 from the syringe 12, the second terminal outlet 24 from the tube 40, and the third terminal inlet 30 from the pressure gauge 14.

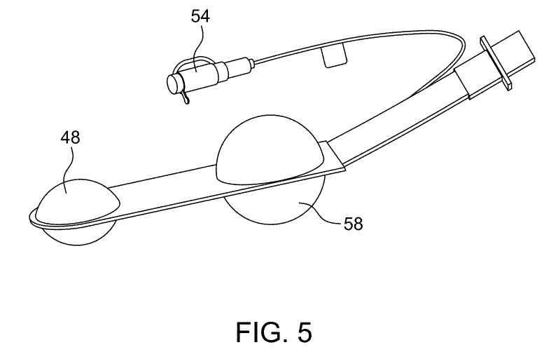

In some embodiments of the presently disclosed subject matter the adapter 16 can be used with a supraglottic, extraglottic, or blind insertion airway device which is shown in FIG. 5. This device can be used to seal the esophagus of a patient. Particularly, in this device, the distal balloon 48 rests in the esophagus and the proximal balloon 58 seals the upper throat or oropharnyx. The device is labeled with an inflation volume, which varies by size, but not a safe inflation pressure. Overfilling this devices has similar or worse deleterious effects as the ET tube.

The adapter 16 can be constructed from any rigid or semi-rigid material known or used in the art including, but not limited to, plastic, rubber, metal, and combinations thereof.

The syringe 12 can be any size known or used in the art, including but not limited to 1 mL-1000 mL. Thus, for example, the syringe can include sizes of at least about (or no more than about) 1, 3, 5, 10, 15, 20, 15, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, or 1000 mL. The syringe 12 may be a luer lock syringe or any syringe known or used in the art.

Other embodiments of the presently disclosed subject matter may include the assembly 10 where the second pathway 26 is positioned relative to the longitudinal pathway 20 at an angle 52 which is greater than zero degrees and less than 180 degrees. Thus, in some embodiments, the angle can be at least about (or no more than about) 1, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, or 179 degrees.

Other embodiments include the assembly 10 where pressure is released from the balloon 42 by actuation of the syringe 12 in a direction opposite from a direction of the original actuation of the syringe 12. In another embodiment the assembly 10 includes a suction port 54 carried by and extending from the tube. In another embodiment the assembly 10 has a pressure gauge 14 which includes a display 56 for displaying a pressure to the user. In another embodiment the assembly 10 has a rigid longitudinal pathway 20 such that movement of the syringe 12 imparts corresponding movement of the adapter 16. In another embodiment of the presently disclosed subject matter the adapter 16 has a second pathway 26 which is positioned relative to the longitudinal pathway 20 at an angle 52 which is greater than zero degrees and less than 180 degrees. The adapter 16 may also have a rigid longitudinal pathway 20 such that movement of the syringe 12 imparts corresponding movement of the adapter 16.

One embodiment of the presently disclosed subject matter may include the assembly 10 where the tube 40 carries a second inflatable balloon 48 that is configured for sealing a patient's esophagus when inflated.

At least one embodiment of the presently disclosed subject matter includes an adapter 16 having a longitudinal pathway 20 that allows for flow through of fluids from a first terminal inlet 22 to a second terminal outlet 24. The adapter 16 may also have a second pathway 26 that allows for flow through of fluids from a third terminal inlet 30 into the longitudinal pathway 20.

In some embodiments, the first terminal inlet 22 defines a threaded portion 32 that selectively engages a syringe 12 when in an operational mode and disengages when in a storage mode. The adapter 16 may have a knurled grip 18 that facilitates stabilization of the adapter 16 when engaged with the syringe 12.

The second terminal outlet 24 selectively engages a tube 40 when in the operational mode that carries an inflatable balloon 42 on an end thereof. The balloon 42 is configured for sealing a patient's trachea 5 when inflated. The tube 40 is disengaged from the second terminal outlet 24 when in the storage mode.

The third terminal inlet 30 selectively engages a pressure gauge 14 when in the operational mode and disengages the pressure gauge 14 when in the storage mode. In the operational mode a user engages the first terminal inlet 22 to the syringe 12, the second terminal outlet 24 to the tube 40, the third terminal inlet 30 to the pressure gauge 14, and the balloon 42 is placed in the patient's trachea 5 and inflated by an original actuation of the syringe 12.

The adapter 16 may have a second pathway 26 positioned relative to the longitudinal pathway 20 at an angle 52 which is greater than zero degrees and less than 180 degrees. The adapter 16 may also have a rigid longitudinal pathway 20 such that movement of the syringe 12 imparts corresponding movement of the adapter 16.

One embodiment of the presently disclosed subject matter includes a method of inflating a balloon 42. The method includes using an adapter 16 that defines a longitudinal pathway 20 which allows for flow through of fluids from a first terminal inlet 22 to a second terminal outlet 24. The adapter also defines a second pathway 26 that allows for flow through of fluids from a third terminal inlet 30 into the longitudinal pathway 20.

The method includes engaging a syringe 12 with a threaded portion 32 at the first terminal inlet 22. The adapter 16 defines a knurled grip 18 that facilitates stabilization of the adapter 16 when engaged with the syringe 12.

The method includes engaging a tube 40 with the second terminal outlet 24. The tube 40 carries an inflatable balloon 42 on an end thereof. The balloon 42 is configured for sealing a patient's trachea 5 when inflated.

The method also includes engaging a pressure gauge 14 with the third terminal inlet 30, placing the balloon 42 in the patient's trachea 5, actuating the syringe 12 to inflate the balloon 42, and checking the pressure of the inflated balloon 42 using the pressure gauge 14.

In some embodiments of the presently disclosed subject matter the method may also include the second pathway 26 positioned relative to the longitudinal pathway 20 at an angle 52 which is greater than zero degrees and less than 180 degrees.

The method may further include determining if pressure needs to be released from the balloon 42, and actuating the syringe 12 in a direction opposite from a direction of the original actuation of the syringe 12 to release pressure from the balloon 42.

The method may also include a suction port 54 carried by and extending from the tube 40. The method may include a pressure gauge 14 having a display 56 for displaying a pressure to the user. The method may also include a rigid longitudinal pathway 20 such that movement of the syringe 12 imparts corresponding movement of the adapter 16.

The method may include a tube 40 carrying a second inflatable balloon 48, wherein the second balloon 48 is configured for sealing a patient's esophagus when inflated.

The method may further include disengaging the syringe 12 from the first terminal inlet 22, disengaging the tube 40 from the second terminal outlet 24, and disengaging the pressure gauge 14 from the third terminal inlet 30 when in the storage mode.

Particular embodiments and features have been described with reference to the drawings. It is to be understood that these descriptions are not limited to any single embodiment or any particular set of features, and that similar embodiments and features may arise or modifications and additions may be made without departing from the scope of these descriptions and the spirit of the appended claims.

What is claimed:

1. An assembly comprising:
a syringe;
a pressure gauge; and
an adapter that defines:
a longitudinal pathway that allows for flow through of fluids from a first terminal inlet to a second terminal outlet;
a second pathway that allows for flow through of fluids from a third terminal inlet into the longitudinal pathway;
wherein the first terminal inlet defines a threaded portion that selectively engages the syringe when in an operational mode and disengages when in a storage mode;
wherein the adapter defines a knurled grip that prevents movement of the syringe when engaged with the threaded portion;
wherein the second terminal outlet selectively engages a tube when in the operational mode that carries an inflatable balloon on an end thereof, wherein the balloon is configured for sealing a patient's trachea when inflated;
wherein the tube is disengaged from the second terminal outlet when in the storage mode;
wherein the third terminal inlet selectively engages the pressure gauge when in the operational mode and disengages the pressure gauge when in the storage mode;
whereby in the operational mode a user engages the first terminal inlet to the syringe, the second terminal outlet to the tube, the third terminal inlet to the pressure gauge, and the balloon is placed in the patient's trachea and inflated by an original actuation of the syringe.

2. The assembly of claim 1, wherein the second pathway is positioned relative to the longitudinal pathway at an angle which is greater than zero degrees and less than 180 degrees.

3. The assembly of claim 1, wherein pressure is released from the balloon by actuation of the syringe in a direction opposite from a direction of the original actuation of the syringe.

4. The assembly of claim 1, further comprising a suction port carried by and extending from the tube.

5. The assembly of claim 1, wherein the tube carries a second inflatable balloon that is configured for sealing a patient's esophagus when inflated.

6. The assembly of claim 1, wherein the pressure gauge includes a display for displaying a pressure to the user.

7. The assembly of claim 1, wherein the longitudinal pathway is rigid such that movement of the syringe imparts corresponding movement of the adapter.

8. An adapter comprising:
a longitudinal pathway that allows for flow through of fluids from a first terminal inlet to a second terminal outlet;
a second pathway that allows for flow through of fluids from a third terminal inlet into the longitudinal pathway;
wherein the first terminal inlet defines a threaded portion that selectively engages a syringe when in an operational mode and disengages when in a storage mode;
wherein the adapter comprises a knurled grip that facilitates stabilization of the adapter when engaged with the syringe;
wherein the second terminal outlet selectively engages a tube when in the operational mode that carries an inflatable balloon on an end thereof, wherein the balloon is configured for sealing a patient's trachea when inflated;
wherein the tube is disengaged from the second terminal outlet when in the storage mode;
wherein the third terminal inlet selectively engages a pressure gauge when in the operational mode and disengages the pressure gauge when in the storage mode;
whereby in the operational mode a user engages the first terminal inlet to the syringe, the second terminal outlet to the tube, the third terminal inlet to the pressure gauge, and the balloon is placed in the patient's trachea and inflated by an original actuation of the syringe.

9. The adapter of claim 8, wherein the second pathway is positioned relative to the longitudinal pathway at an angle which is greater than zero degrees and less than 180 degrees.

10. The adapter of claim 8, wherein the longitudinal pathway is rigid such that movement of the syringe imparts corresponding movement of the adapter.

11. A method of inflating a balloon comprising:
providing an adapter that defines:
a longitudinal pathway that allows for flow through of fluids from a first terminal inlet to a second terminal outlet; and
a second pathway that allows for flow through of fluids from a third terminal inlet into the longitudinal pathway;
engaging a syringe with a threaded portion at the first terminal inlet, wherein the adapter defines a knurled grip that facilitates stabilization of the adapter when engaged with the syringe;
engaging a tube with the second terminal outlet, the tube carrying an inflatable balloon on an end thereof, wherein the balloon is configured for sealing a patient's trachea when inflated;
engaging a pressure gauge with the third terminal inlet;
placing the balloon in the patient's trachea;
actuating the syringe to inflate the balloon; and
checking the pressure of the inflated balloon using the pressure gauge.

12. The method of claim 11, wherein the second pathway is positioned relative to the longitudinal pathway at an angle which is greater than zero degrees and less than 180 degrees.

13. The method of claim 11, further comprising:
determining if pressure needs to be released from the balloon; and
actuating the syringe in a direction opposite from a direction of the original actuation of the syringe, thereby releasing pressure from the balloon.

14. The method of claim 11, further comprising a suction port carried by and extending from the tube.

15. The method of claim 11, wherein the pressure gauge includes a display for displaying a pressure to a user.

16. The method of claim 11, wherein the longitudinal pathway is rigid such that movement of the syringe imparts corresponding movement of the adapter.

17. The method of claim 11, wherein the tube carries a second inflatable balloon that is configured for sealing a patient's esophagus when inflated.

18. The method of claim 11, further comprising disengaging the syringe from the first terminal inlet when in a storage mode.

19. The method of claim 11, further comprising disengaging the tube from the second terminal outlet when in a storage mode.

20. The method of claim 11, further comprising disengaging the pressure gauge from the third terminal inlet when in a storage mode.
